(19) United States
(12) Patent Application Publication
(10) Pub. No.: US 2025/0222192 A1
(43) Pub. Date: Jul. 10, 2025

(54) FLUID DETECTION IN IRRIGATION SYSTEM

(71) Applicant: Coloplast A/S, Humlebaek (DK)

(72) Inventors: Niels Hvid, Vedbaek (DK); Benjamin Holm Salka, Copenhagen N (DK)

(21) Appl. No.: 18/853,451

(22) PCT Filed: Mar. 29, 2023
(86) PCT No.: PCT/DK2023/050068

§ 371 Date: Oct. 2, 2024

(30) Foreign Application Priority Data
Apr. 8, 2022 (DK) PA202270190

(51) Int. Cl. A61M 3/02

(52) U.S. Cl. CPC A61M 3/0208; A61M 3/0237; A61M 3/0258; A61M 2205/3317; A61M 2205/50; A61M 2210/1064

(57) ABSTRACT

An irrigation system is disclosed. The system comprises a housing comprising a reservoir for containing an irrigation liquid; a tubing providing fluid communication between the reservoir and a catheter for use with the irrigation system; a pump for facilitating a flow of irrigation liquid from the reservoir, through the tubing, to the catheter; and a control unit comprising a processor in communication with the pump; a capacitive sensor in communication with the processor and configured to measure one or more capacitive characteristics of a fluid in the tubing. The processor is configured to control the irrigation procedure in accordance with the one or more capacitive characteristics of the fluid in the tubing. Also disclosed is an associated method.

The present disclosure relates to an irrigation system for irrigation of the bowels of a user, the system comprising a capacitive sensor for measuring one or more capacitive characteristics of a fluid in a tubing of the system. Further, the disclosure relates to a method of controlling an irrigation procedure.

### BACKGROUND

Bowel irrigation is one of a number of treatments used to aid people with bowel problems. People suffering from bowel problems are often paralyzed, typically due to spinal cord injuries, and confined to a wheelchair or hospitalized. In these situations, often the peristaltic functions, i.e., the reflexes and muscles of the bowel, cannot be stimulated correctly. This results in constipation or random discharge of bowel contents. By using bowel irrigation, a stimulation of the peristaltic movements of the colon can be provided. To perform such bowel irrigation, a device comprising a catheter, also referred to as an anal catheter, anal probe, rectal catheter, or speculum, is provided. The catheter is inserted into the rectum through the anus. A liquid, also referred to as an irrigation liquid, such as water or a saline solution, is then introduced into the rectum/bowels through the catheter. The amount of liquid is generally up to 1.5 litres, depending on the person. The introduced liquid stimulates the peristaltic movements of the bowel. After a specified period of time, such as 15 minutes, the catheter is removed, and the liquid, along with output from the bowel, is released through the anus.

### BRIEF DESCRIPTION OF THE DRAWINGS

The accompanying drawings are included to provide a further understanding of embodiments and are incorporated into and a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 illustrates an exemplary irrigation system according to an embodiment of the invention;

FIG. 2 illustrates an exemplary cross-sectional view of a housing of an irrigation system according to an embodiment of the invention;

FIG. 3 illustrates a capacitive sensor according to an embodiment of the invention;

FIG. 4 illustrates a capacitive sensor according to an embodiment of the invention;

FIG. 5 illustrates a circle and two tangent planes thereof being mutually parallel;

FIG. 6 illustrates an exemplary capacitive sensor according to an embodiment of the invention;

FIG. 7 illustrates an exemplary graph depicting the capacitance as a function of time;

FIG. 8 illustrates an exemplary graph depicting the capacitance as a function of time;

FIG. 9 illustrates a method of controlling an irrigation procedure in an irrigation system according to embodiments of the invention; and

FIG. 10 illustrates a method of controlling an irrigation procedure in an irrigation system according to embodiments of the invention.

### DETAILED DESCRIPTION

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

In the following, whenever referring to a proximal end of an element of the invention, the referral is to the end adapted for insertion. Whenever referring to the distal end of an element, the referral is to the end opposite the insertion end. In other words, the proximal end is the end closest to the user, when the catheter is to be inserted and the distal end is the opposite end—the end furthest away from the user when the catheter is to be inserted. The longitudinal direction is the direction from the distal to the proximal end. The transverse direction is the direction perpendicular to the longitudinal direction, which corresponds to the direction across the shaft of the catheter.

In the following, whenever referring to a bowel irrigation system or bowel irrigation, the referral to a system or method capable of irrigating the bowels of a user using a catheter. Commonly, the catheter is inserted through the anus. Bowel irrigation (systems) is also known in the art as anal irrigation (systems) and rectal irrigation (systems), and the terms may be used interchangeably in embodiments where the bowel irrigation system is adapted for use through the anus.

In the following, whenever referring to the bowel(s) of a user, the referral is to the intestines of the user. The referral can be to the lower intestines specifically, e.g., the rectum and/or the colon/large intestine. In the following, whenever referring to the rectum, the referral is to the terminal section/canal of the intestine ending in the anus. In the following, whenever referring to the anus, the referral is to the opening of the lower end of the alimentary canal, through which refuse of digestion is commonly excreted. In the following, whenever referring to anal, the referral is to a feature, device, method, or system pertaining to the anus, e.g., pertaining to engagement with or through the anus. In the following, whenever referring to the rectal walls, the referral is to the intestinal wall surrounding and defining the canal of the rectum.

In the following, whenever referring to a quantity, such as a capacitive characteristic, being in compliance with a threshold value, the referral is to the quantity being within the range where such quantity attains a desired value. Where the threshold value defines an upper limit to an acceptable value of the quantity, compliance is fulfilled whenever the value is equal to or below the threshold value. Likewise, where the threshold value defines a lower limit to an acceptable value of the quantity, compliance is fulfilled whenever the value is equal to or above the threshold value. Accordingly, non-compliance is used to describe the value of the quantity not being in compliance, i.e., falling outside the scope of compliance.

In the following, whenever referring to a fluid, the referral is to any liquid, gas or other material that continuously deforms under an applied shear stress, or external force, as defined in fluid dynamics.

In particular, within the present invention, the term “fluid” includes both the liquid and gas phases. In other words, a fluid may be a liquid or a gas. In the following, whenever referring to a liquid, the referral is to a (nearly) incompressible fluid that conforms to the shape of its container but retains a (nearly) constant volume independent of pressure. In the following, whenever referring to a gas, the referral is to a fluid that has neither independent shape nor volume. In particular, within the present invention, the gas may be (ambient) air, unless otherwise specified. Thus, “gas” and “air” may be used interchangeably.

The present disclosure provides an irrigation system configured to provide an irrigation procedure for irrigation of the bowels of a user and a method of controlling an irrigation procedure in, or a pump of, an irrigation system.

In a first aspect of the invention, an irrigation system is disclosed. The irrigation system is for running an irrigation procedure for irrigation of the bowels of a user. In other words, the irrigation system is for irrigation of the bowels of the user, and in particular for irrigation of the bowels of a user via employment of an irrigation procedure as provided by the system. The system comprises:
a housing comprising a reservoir for containing an irrigation liquid;
a tubing providing fluid communication between the reservoir and a catheter for use with the irrigation system;
a pump for facilitating a flow of irrigation liquid from the reservoir, through the tubing, to the catheter;
a control unit comprising a processor in communication with the pump; and
a capacitive sensor in communication with the processor and configured to measure one or more capacitive characteristics of a fluid in the tubing.

The processor may be configured to control the irrigation procedure (e.g., to control the pump) in accordance with the one or more capacitive characteristics of the fluid in the tubing. The at least one processor may be configured to control the pump in accordance with the one or more capacitive characteristics of the fluid in the tubing. The at least one processor may be configured to control a valve associated with the tubing in accordance with the one or more capacitive characteristics of the fluid in the tubing. In an embodiment, to control the irrigation procedure comprises to control the pump, such as the operation of the pump. In the following, a reference to “control the irrigation procedure” may be a reference to “control the pump” or a reference to “control the valve”. The reference “to control the irrigation procedure” is used for simplicity, but it is appreciated that an irrigation procedure may be performed even without association with a human body. In particular, it is appreciated that “to control the irrigation procedure” may be embodied by controlling the pump, a valve, or another element or feature of the irrigation system.

The system may comprise a catheter for arrangement in a user. The catheter may be coupled, such as releasably coupled, to an end of the tubing.

The irrigation system may be said to be configured to run or provide an irrigation procedure, whereby it is meant that the irrigation system is configured to, by means of the features of the system as disclosed, to irrigate the bowels of a user, said irrigation of the bowels of the user being the irrigation procedure.

By the irrigation system being configured to run an irrigation procedure is meant that the irrigation system is configured to, by means of the features of the system as disclosed, provide a process resulting in, or supporting, irrigation of the bowels of a user. For example, running an irrigation procedure may include pumping irrigation liquid from the reservoir to the rectum of a user, said pumping of irrigation liquid being performed in an irrigation system as disclosed.

The irrigation system may be a portable irrigation system. By a portable irrigation system is meant a system having a size and weight suitable for carry by a user, such that the user may use the system in various settings and in various places. For example, the irrigation system may have means for operating without external power, such as by means of a (rechargeable) battery/power unit. In embodiments, the battery is rechargeable via a charging interface of the housing of the irrigation system. In an alternative embodiment, the irrigation system is powered by means of a wired connection to an outlet of a power grid.

The housing comprises a reservoir. The reservoir may be any reservoir suitable for holding a liquid. In embodiments, the reservoir can be considered a container. In embodiments, the reservoir is capable of storing at least the amount of liquid required for an irrigation procedure. The reservoir is sealable, such as to avoid spillage. In embodiments, the reservoir is hermetically sealable such that fluids may not escape the reservoir through other openings than that of the tubing.

In embodiments, the irrigation liquid is water, such as tap water, or a saline solution. In embodiments, the provision of liquid can be through a simple procedure of gaining access to the reservoir, e.g., by opening or removing a lid of the housing, and pouring liquid into the reservoir.

The housing may further comprise an electronics compartment sealed from the reservoir, the electronics compartment comprising electronic components and/or sensors. In a preferred embodiment, the electronics compartment is arranged in a lid configured to close off the reservoir. The electronics compartment may comprise electronics associated with the irrigation system, such as a powering circuit, including a power unit/battery, the control unit, and the pump. The electronics compartment may further comprise additional elements cooperating with, connecting, or connected to, parts of the control unit, and/or the pump. The electronics compartment may be vented relative to ambient air, such as through a filter. The electronics compartment is preferably sealed relative to the reservoir such that liquid or gasses from the reservoir cannot escape into the electronics compartment. The electronics compartment may further comprise one or more sensors connected to at least the processor. The one or more sensors may comprise one or more pressure sensors. The one or more pressure sensors may be a relative pressure sensor configured to measure the pressure in the reservoir relative to the electronics compartment and sending corresponding data to the processor and/or memory of the control unit. The one or more pressure sensors may be an absolute pressure sensor configured to measure the absolute pressure in the reservoir and sending corresponding data to the processor and/or memory of the control unit.

In embodiments, the electronics compartment is contained in a lid of the reservoir. In embodiments, the housing comprises the reservoir and a lid, wherein the lid comprises the electronics compartment.

In embodiments, the irrigation system comprises a catheter. A catheter may also be denoted an anal probe in the art. In embodiments the catheter is provided with eyelets in the proximal end, the eyelets communicating with an irrigation channel inside the catheter, so that irrigation liquid pumped into the catheter in a distal end can exit the catheter through the eyelets in the proximal end. Tests have shown that a diameter of the irrigation channel of approximately 3-7 mm, for example 4.3 mm, allows an adequate flow. The catheter may comprise retention means for securely arranging the catheter in the anus of the user. Retention means may comprise an inflatable balloon.

In embodiments, the catheter is adapted for insertion into a rectum of the user and for arrangement in the user. One useful exemplary catheter comprises a main tubular part, typically called a shaft, extending from the distal end to the proximal end. A tip is positioned in the proximal end of the catheter and is provided as a rounded closed end of the shaft. In embodiments, the catheter comprises a connector in the distal end and may in an embodiment comprise a flared end of the catheter so that the diameter of the connector increases with respect to the tubular part. In particular, the proximal end of the catheter is configured for insertion into the rectum of the user. Usually, catheters used for bowel irrigation are 8-16 mm in external diameter, for example 10 mm. The length can be 70-200 mm, for example 150 mm. In embodiments, the catheter is of a size reflecting the needs or requirements of the user. As such, a range of different catheter sizes can be provided.

In embodiments, the catheter is adapted for insertion by means of having an appropriate size, shape, and through an appropriate material choice. In embodiments, a method of bowel irrigation comprises the step of inserting the catheter into a rectum of a user. In embodiments, insertion of the catheter into the rectum is carried out manually by the user or by a health care professional. In embodiments, insertion is aided by the provision of a lubricant. In embodiments, insertion is aided by a certain surface treatment of the catheter reducing its friction. In embodiments, the catheter is inserted by a distance such that at least the proximal end with eyelets is past an anal sphincter of the user, such as the internal anal sphincter.

The irrigation system comprises a tubing providing fluid communication between the reservoir and a catheter for use with the irrigation system. In embodiments the provision of a tubing connecting the reservoir and a catheter facilitates transfer of liquid from the reservoir to the rectum via eyelets in the proximal end of the catheter and therefrom into the rectum once inserted. In embodiments, the tubing comprises at least one lumen extending from a first end to a second end of the tubing. In embodiments, the first end of the tubing extends into the reservoir, or is arranged in communication with the reservoir, e.g., via a channel in the housing of the irrigation system, such that irrigation liquid contained in the reservoir may enter the tubing via the first end. In particular, in embodiments, the first end of the tubing may extend to, or be arranged within or in proximity to, a bottom of the reservoir. Here, the bottom of the reservoir denotes the part of the reservoir where liquid will accumulate when the housing is arranged on a plane surface and exposed to gravity. In other words, the first end of the tubing may be configured to extend into the irrigation liquid when such liquid is arranged/contained in the reservoir. The first end may form part of a dip-tube configured to extend into the irrigation liquid when such liquid is arranged/contained in the reservoir. In embodiments (as discussed later), where the pump is an air pump configured to pressurize the reservoir, such pressurization may cause liquid to be forced into the tubing due to the provision of the dip tube or tubing extending into the irrigation liquid.

In embodiments, the second end of the tubing comprises a connector configured to connect/mate with a catheter. In embodiments where retention means of the catheter comprises a balloon, the tubing may comprise two lumens including a first and a second lumen, the first lumen connecting the interior of the balloon and the reservoir, and the second lumen connecting the tip of the catheter and the reservoir. In embodiments, the tubing is flexible. In embodiments, at least parts of the tubing are transparent for providing visual assessment of the flow of fluid.

In embodiments, the tubing is integral from the first end to the second end. In a preferred embodiment, the tubing comprises two or more segments including a dip tube arranged in the reservoir, a flexible tubing segment extending from the housing to the catheter, and a channel or intermediate tube section/segment connecting the dip tube and the flexible tubing segment. For example, the intermediate tube section may be a channel provided in the housing and comprising a connector for connecting the flexible tubing segment to the housing and thus the dip tube.

In embodiments, a first part of the tubing is flexible, and a second part of the tubing is rigid. In embodiments, the first part extends from the housing to the second end of the tubing, and the second part is formed as a channel in, or integrated part of, the housing. In embodiments, the dip-tube is flexible. In embodiments, the dip-tube is rigid. In embodiments, the tubing comprises the dip tube, the intermediate tube section, and a flexible tubing comprising a connector for connecting to a catheter. The intermediate tube section may be arranged in the lid such that fluids may enter the dip tube and flow through said dip tube, through the intermediate tube section of the lid and further into the flexible tubing connected to a catheter. Thus, by “tubing” is meant a pathway or lumen providing fluid communication between the first end and the second end, and such pathway or lumen may be partly rigid and formed as part of the housing, and partly flexible and extending out of the housing. In other words, when a tubing is discussed in the following, the reference is to a tubing providing fluid communication between the reservoir and a catheter for use with the irrigation system, and it is appreciated that such tubing may comprise multiple elements which, when assembled, provides such fluid communication through a lumen.

The irrigation system comprises a control unit. The control unit may be built into the housing, such as contained in a part of the housing separate from the reservoir, such as in an electronics compartment. In a preferred embodiment, the control unit is contained in a lid for the reservoir. The control unit comprises at least one processor. The control unit may comprise a memory connected to the processor and an interface allowing connection to the at least one processor. The control unit may comprise one or more buttons including a power button for turning the irrigation system on/off. The control unit may comprise a button for turning the pump on/off.

In embodiments, the control unit comprises a user interface for receiving inputs from the user and/or providing outputs to the user. For example, the user interface may be a graphical user interface for presenting visual information to the user. The user interface may be provided in a surface of the lid of the irrigation system where the control unit is provided in such lid. The control unit may comprise one or more indicators, such as light indicators, such as LED's, for communicating an operating status of the irrigation system. The control unit may comprise means for providing an audible signal. In embodiments, the audible signal is provided by adequate control of the pump for generating a sound originating from the pump mechanics. The control unit may comprise means for providing a haptic feedback.

The control unit may comprise a remote, such as a handheld remote configured to control the irrigation system via wired or wireless means. The remote may comprise a second processor and/or memory. The remote may be an accessory control unit configured to communicate with a control unit built into the housing. In embodiments, the user interface is provided in the remote. The remote may comprise a second processor and/or memory. The remote may be an accessory control unit configured to communicate with a control unit built into the housing.

In embodiments, the control unit is configured to communicate with a lumen of the tubing, such that the control unit can assess the fluid flowing within the lumen of the tubing. In embodiments, an electrical wiring is provided within the electronics compartment to provide an electrical connection between the pump and the control unit, such that the control unit may, or can be used to, control the performance of the pump and/or other parts of the irrigation system. In embodiments, an electrical wiring is provided between the catheter and the control unit, e.g., via the tubing, thereby providing electrical communication therebetween, such as where the catheter comprises one or more sensors. In embodiments, the control unit and the pump are in communication according to a wireless protocol.

The irrigation system comprises a pump facilitating a flow of irrigation liquid from the reservoir, through the tubing, to the catheter. The pump may be controllable by the processor. The pump is connected to a power source, such as a power unit comprising a battery. The power source provides an operating voltage to the pump. The power source may further power further electronics of the system, such as the processor(s) and the sensor(s).

In a preferred embodiment, the pump is an electrical pump. In an embodiment, the pump is an electrical air pump configured to pressurize the reservoir to facilitate the flow of irrigation liquid. The flow of irrigation liquid may be facilitated by means of the dip tube of the tubing extending into the irrigation liquid in the reservoir, whereby the pressurization of the (air of the) reservoir forces liquid in the reservoir into and through the tubing and towards a catheter attached to said tubing.

In embodiments, the pump is powered by a power unit of the irrigation system, such as via a battery contained in the housing, such as in the electronics compartment. In embodiments, the battery is rechargeable via a charging interface of the housing of the irrigation system. In embodiments, the irrigation system is powered by means of a wired connection to an outlet of a power grid. In a preferred embodiment, the irrigation system is a portable irrigation system, whereby the user may bring the irrigation system to a desired location to perform the irrigation procedure.

The pump facilitates a flow of fluid, in particular of irrigation liquid, in/through the tubing. In a preferred embodiment, the pump is configured to pressurize the reservoir by means of increasing the air pressure inside the reservoir where the reservoir is partly filled with liquid. For example, the pump may be an electrical air pump configured to pump air into the reservoir by taking in ambient air, such as via a vent in the housing in communication with the pump. In a preferred embodiment, the reservoir is hermetically sealable. In a preferred embodiment, the pressurization of the reservoir causes liquid to escape the reservoir via the tubing, in particular where the tubing extends into the liquid, e.g., by means of the dip-tube as discussed previously. For example, where the housing, and thus the reservoir, is provided with a preferred bottom upon which the user is expected to place the housing on a plane surface, the first end of the tubing may be arranged near this bottom. Thereby, when the irrigation system is prepared for irrigation, liquid will cover the first end/entrance of the dip tube and thus the tubing, and when the air pressure inside the reservoir is increased, the liquid will be forced into the tubing and further into the rectum when a catheter connected to the tubing is inserted in the rectum of a user.

In embodiments, liquid automatically starts to flow through the tubing once an adequate pressure has been reached (taking into account, e.g., gravitational effects of the arrangement of the reservoir relative to the end (catheter) of the tubing) and continues to do so until either the reservoir has been emptied, the dip tube of the tubing is not covered with liquid, the pressure is not sufficient to provide a flow of liquid, or the pump is turned off.

In alternative embodiments, the pump may be a gear pump or a centrifugal pump. In embodiments, the pump is a reversible electrical pump. In embodiments, the pump is able to pump air through the system in order to empty the tubing from liquid.

In embodiments, the capacitive sensor is configured to measure a capacitive characteristic of a fluid in the tubing. In embodiments, the capacitive sensor is configured to measure one or more capacitive characteristics of a fluid in the tubing. In an embodiment, the one or more capacitive characteristics are selected from one or more of (i) a capacitance as induced by a fluid, (ii) a permittivity of a fluid, (iii) a change of the capacitance as induced by a fluid, and (iv) a change of the permittivity. A capacitive characteristic may also be denoted a parameter, such as a capacitive parameter, implying the nature of the parameter being capacitance or associated with capacitance.

Preferably, the capacitive sensor is arranged to measure the one or more capacitive characteristics of a fluid in the tubing. In a preferred embodiment, the capacitive sensor is configured to detect presence of liquid and/or gas in the tubing based on analysis of the one or more capacitive characteristics as measured. For example, the capacitive sensor may be configured to detect a change from liquid being present in the tubing (in vicinity of the capacitive sensor and thus causing a certain capacitive characteristic to be measured, such as a first capacitance) to gas being present in the tubing (in vicinity of the capacitive sensor and thus causing another certain capacitive characteristic to be measured, such as a second capacitance different from the first capacitance), and vice versa. The change of the capacitive characteristic being measured (e.g., capacitance) may be detected by the capacitive sensor as such, such as via an auxiliary processor contained in the capacitive sensor, or the change of capacitance may be detected by the processor of the control unit. Thus, the capacitive sensor may be specialised in distinguishing/differentiating specifically liquids, such as irrigation liquid, such as water, and gasses, such as ambient air. Thereby, the capacitive sensor may be specifically adapted for use with an irrigation system where liquid and gas are the primary materials flowing in the tubing.

By a capacitive sensor is meant a sensor configured to measure a capacitance or derivative thereof. In other words, the capacitive sensor is configured to measure one or more capacitive characteristics of a dielectric medium (e.g., a fluid) in proximity to the capacitive sensor, such as between one or two elements constituting the plates of a plate capacitor. Thus, in embodiments, the capacitive sensor comprises a capacitor arranged to measure one or more capacitive characteristics of a tubing and a fluid therein. By a capacitive characteristic is meant any electrical quantity or value associated with, or derivable from, a capacitor, including the absolute value of capacitance, a change of capacitance over time, the absolute value of the (relative) permittivity of a fluid, or a change of (relative) permittivity (e.g., as caused by a change in the dielectric medium in proximity to the capacitive sensor over time). The capacitive sensor may be configured to continuously monitor the one or more capacitive characteristics of the fluid in the tubing, such as at a frequency higher than 0.1 Hz, such as higher than 1 Hz, such as higher than 10 Hz or higher than 100 Hz.

In general, the capacitance C of a parallel-plate capacitor may be given as:

$$C = ε \frac{A}{d} = {ε}_{r} {ε}_{0} \frac{A}{d}$$

wherein ε is the permittivity of the (dielectric) medium between the parallel plates, A is the area of each of the parallel plates, and d is the separation of the parallel plates, said separation being a gap filled with the (dielectric) medium. The permittivity E may be expressed as the product of the relative permittivity εᵣ of the medium and the vacuum permittivity ε₀. As such, a medium having a permittivity E may be said to induce a capacitance when the area A and the separation d are constant. The vacuum permittivity ε₀ is a physical constant having the value 8.8541878128×10⁻¹² F m⁻¹, and the relative permittivity εᵣ of water is 80.2 (at 20° C.) and the relative permittivity of air is 1.00058986. For vacuum, the relative permittivity is one by definition. Thus, the relative permittivity of water is approximately 80 times the relative permittivity of air, which translates to a similar change of the size of the capacitance of the capacitive sensor when a change from liquid to air occurs in the tubing in proximity to the capacitive sensor and vice versa.

Whereas embodiments of the capacitive sensor of the present invention may deviate from that of a parallel-plate capacitor, the theory of the parallel-plate capacitor is here used to highlight the overall relationship between permittivity (material dependent) and geometrical factors of the build of the capacitive sensor (area and separation). Thus, the present discussion refers to the above relationship for the capacitance of a parallel-plate capacitor, and it is appreciated that the discussion may be applied to other builds of capacitors wherein the capacitance may be calculated differently according to the geometry of such different builds. In other words, whereas not all embodiments of the capacitive sensor according to the present invention may be modelled as a parallel-plate capacitor, the model may be used to express the general relation between capacitance, permittivity, and geometry of the capacitor.

In embodiments, when a fluid passes through a plate capacitor (e.g., through a tubing arranged between the plates of the capacitor), the capacitance is dependent on (is induced by) the permittivity of said fluid. For example, when the fluid is water, the capacitance is dependent on the relative permittivity for water, and when the fluid is air, the capacitance is dependent on the relative permittivity for air. As stated above, the relative permittivity of water is approximately 80 times the relative permittivity of air, and as such, the change of capacitance is significant when the characteristics (e.g., the presence of air bubbles in the flow of water) of the fluid changes over time. The permittivity of the material (e.g., plastic) of the tubing as such, arranged in vicinity of the capacitor, may be neglected as it may be fixated relative to the capacitive sensor, and thus does not vary over time.

In other words, the capacitance over time C(t) is dependent on the changes of permittivity (of the fluid in the tubing) over time ε(t) in the capacitor:

$$C ( t ) = ε ( t ) \frac{A}{d} = {ε}_{r} ( t ) {ε}_{0} \frac{A}{d}$$

Thereby, if air (e.g., occasional bubbles, or due to the reservoir being emptied) appear in the tubing during an irrigation procedure wherein liquid is passing by/through the capacitor, the capacitive sensor may output/send a signal to the processor of the irrigation system, the signal being indicative of air in the tubing, and said processor may control the irrigation procedure, such as the pump, in accordance with such signal, or more generally; in accordance with the capacitive characteristics of the fluid in the tubing.

The capacitive sensor may be in communication with the processor of the control unit. By the capacitive sensor being in communication with the processor is meant that the processor may receive inputs from the capacitive sensor, or the processor may be configured to control the capacitive sensor. For example, the processor may be configured to control other parts of the irrigation system based on measurements/readings by the capacitive sensor.

The processor may be configured to receive data indicative of the one or more capacitive characteristics from the capacitive sensor. The processor may be configured to control the irrigation procedure (e.g., the pump) in accordance with the one or more capacitive characteristics (as received from the capacitive sensor) of the fluid in the tubing. In embodiments, the processor is configured to control the pump in accordance with the one or more capacitive characteristics of the fluid in the tubing. For example, the processor may turn on/off the pump in accordance with the capacitive characteristics. In embodiments, the processor is configured to control pressurization of the reservoir in accordance with the capacitive characteristics of the fluid in the tubing.

By control is meant that the processor may be configured to influence, regulate, or affect the irrigation procedure in accordance with the measured one or more capacitive characteristics. For example, in other words, the processor may take a capacitive characteristic as input and generate an output comprising instructions on how to operate the pump based on said capacitive characteristic. For example, the output may comprise instructions to power off the pump in accordance with/based on the measured capacitive characteristic. To control may comprise to terminate, initiate, pause, resume/reinitiate the irrigation procedure, or to disable irrigation system as such.

In embodiments, the processor is configured to control the irrigation procedure, such as to terminate the irrigation procedure, such as by means of turning the pump off, in response to the one or more capacitive characteristics of the fluid in the tubing being indicative of air.

In embodiments, the processor is configured to control the irrigation procedure, such as to (re-) initiate the irrigation procedure, such as by means of turning the pump on, in response to the one or more capacitive characteristics of the fluid in the tubing being indicative of liquid, such as irrigation liquid.

For example, the processor may determine the type of fluid in the tubing at any given time based on the one or more capacitive characteristics, including whether liquid or gas (air) is present in the tubing, and therefrom, the processor may be configured to turn off the pump in accordance with the capacitive characteristics of the fluid being indicative of air in the tubing.

The processor may be configured to receive an input, such as a binary input, from the capacitive sensor being indicative of liquid and/or gas in the tubing. For example, the capacitive sensor may be configured to provide a binary output in accordance with the capacitive characteristics of the fluid in the tubing. For example, the binary output may be provided in accordance with the considered capacitive characteristic (e.g., the capacitance) being above or below a defined threshold value. Thereby, there need not to be a determination of the absolute value of the capacitive characteristic as such, but merely a determination of its compliance with a defined threshold value.

In an alternative embodiment, the capacitive sensor as such may be configured to control the pump, and as such aspects of the irrigation procedure, in accordance with the capacitive characteristics of the fluid in the tubing. Namely, in such alternative embodiment, the capacitive sensor may be coupled to the electrical pump via a transistor (logic gate), where the transistor is configured to prevent provision of an operating voltage to the pump. In other words, by proper control of a logic gate, such logic gate may effectively turn off the pump in response to a binary (logic) output from the capacitive sensor as such being indicative of air in the tubing. For example, the transistor may be configured as an AND-gate. Thereby, the pump may be operated independently of a processor of the irrigation system. Thereby, the irrigation system may be simplified. Thus, whereas in the majority of embodiments described herein, a processor of the control unit receives the output from the capacitive sensor and said processor controls the irrigation procedure, which may include control of the pump, it is appreciated that such irrigation system may be simplified further by connecting the capacitive sensor to a logic gate connected to a voltage source for the pump and to the pump. In other words, it is appreciated that some functionality described herein may be carried out by the capacitive sensor in combination with a transistor (logic gate), in particular where the capacitive sensor is configured to provide a binary output in response to the capacitance as measured, which binary output may serve as an input for a transistor arranged in series with a voltage to the pump

By being configured to control the irrigation procedure, such as to terminate/pause the irrigation procedure (e.g., to turn off the pump), in accordance with the one or more capacitive characteristics of the fluid in the tubing, the system may quickly respond to changes, and the pump may be turned off to avoid any further pumping and/or pressure increase in the reservoir and/or the user. For example, turning off the pump may cause the irrigation system to enter a safety mode wherein further pumping of liquid is avoided, while at the same time avoid the pump to increase the pressure in the reservoir (e.g., where the pump is an air pump configured to provide an increased air pressure in the reservoir).

An irrigation system according to the first aspect of the invention provides for an automated pumping of liquid including a mechanism allowing for termination of the irrigation procedure in case of gas (air) being pumped into the tubing and further into the user—for example due to faulty operation or the reservoir being empty of liquid.

Further, the disclosed use of a capacitive sensor provides for a non-invasive mechanism for analysis of the fluid in the tubing, such that said tubing may be provided in one piece and/or completely sealed, and such that no sensors are to be accommodated within the tubing or have exposed sensing faces in contact with a liquid, which may cause corrosion. Capacitive sensing may be understood as being inherently non-invasive due to its dependability on the immediate surroundings of the sensor rather than on a physical interaction between media (e.g., between a sensor surface and the medium to be detected). Further, the use of a capacitive sensor provides for easy maintenance and cleaning of the tubing, as the capacitive sensor is decoupled (non-invasive) from the tubing as such: the capacitive sensor may be provided adjacent to the tubing, such as along/tangential to an outer surface of the tubing. In other words, the capacitive sensor acts as a proximity sensor configured to sense changes in the proximity to, for example, a sensor surface of the capacitive sensor. Thus, the tubing may not need specific arrangements or adaptions for working with the capacitive sensor according to the first aspect of the invention.

In further other words, embodiments of the present aspect of the invention provide a mechanism configured to determine the nature of fluid in the tubing. Further, a mechanism configured to respond to such nature of fluid in the tubing is provided. Thus, embodiments of the present aspect provide means to detect whether the reservoir is empty of irrigation liquid, in which case air may start to flow through the tubing, which is undesirable for both the system and the user. Thereby, it may be avoided that the irrigation system is running despite the reservoir being emptied of irrigation liquid. Likewise, it may be avoided that the user experiences air being pumped into his/her rectum due to either the presence of air bubbles or due to the reservoir being empty of irrigation liquid.

In embodiments, the system comprises a valve in communication with the tubing and preferably the control unit, the valve being configured to control the flow of fluid in the tubing. In embodiments, the processor is configured to control a valve associated with the tubing in accordance with the one or more capacitive characteristics of the fluid in the tubing. For example, in embodiments, the valve is controllable between a closed state, thereby preventing a flow of fluid through the tubing, and a fully open state, thereby providing an unrestricted flow of fluid through the tubing. In embodiments, the processor is configured to close the valve in response to the capacitive characteristics of the fluid being indicative of air. Such control of a valve in accordance with the capacitive characteristics of the fluid in the tubing may provide for quick adjustments to the irrigation procedure, wherein the valve may close during an assessment of whether further irrigation is safe. In the affirmative of the latter, the valve may re-open, and the irrigation procedure may continue.

Alternatively, similar to what is disclosed above, the capacitive sensor may operate a valve independently (e.g., from a processor) by means of a transistor/logic gate coupled to the capacitive sensor and the valve. To control the irrigation procedure may comprise to control the valve.

In an embodiment, the processor is configured to terminate the irrigation procedure in accordance with the one or more capacitive characteristics of the fluid in the tubing being indicative of air.

To terminate may mean to terminate completely (i.e., no further irrigation is possible for the present procedure (e.g., in the case of the reservoir being empty of irrigation liquid) and/or future irrigation procedures) or to terminate intermittently/pause, such as to allow the system to reset or the user to provide an input.

In embodiments, the processor is configured to terminate the irrigation procedure in accordance with/in response to the characteristics of the fluid in the tubing being indicative of air for a time period exceeding a first threshold. Thus, the processor may be configured to terminate the irrigation procedure in response to the characteristics of the fluid in the tubing being indicative of air after X seconds after first measuring a capacitive characteristic being indicative of air, where X seconds may be selected between 10 s and 60 s (see below for further discussion).

For example, if the one or more capacitive characteristics of the fluid in the tubing are indicative of air for a certain time period (e.g., following a period of the characteristics of the fluid in the tubing being indicative of liquid, meaning that the irrigation procedure is ongoing), the processor may be configured to terminate the irrigation procedure, such as to turn off the pump and/or close a valve. For example, air may become present in the tubing once the reservoir is emptied of irrigation liquid. However, due to an internal volume of the tubing, pressurization/pumping of liquid may continue for a certain period of time during which said internal volume of the tubing is emptied of liquid. For example, the first threshold may be set according to an internal volume of the tubing and/or a pumping speed of the irrigation system. For example, in embodiments, the pumping speed may be 250 ml/min and the internal volume of the tubing may be 100 ml. In such a case, and in particular when the capacitive sensor is arranged in vicinity of the first end of the tubing where fluid enters said tubing, the pump may continue operation for 24 s (100 ml/250 ml/min) after air has been detected by the capacitive sensor without air being pumped into the user in the second end of the tubing where fluid exits the tubing via a catheter. Thus, the first threshold may between 10 s and 60 s, such as between 15 s and 45 s, such as between 20 s and 30 s. In embodiments, the first threshold is defined by an internal volume of the tubing (e.g., given in ml) divided by a pumping speed of the irrigation system (e.g., given in ml/min). In embodiments, the internal volume of the tubing is between 50 ml and 200 ml, such as between 75 ml and 150 ml, such as 100 ml. In embodiments, the pumping speed is between 100 ml/min and 500 ml/min, such as between 200 ml/min and 300 ml/min, such as 250 ml/min.

By allowing air to be present in the tubing for a certain time period after the capacitive sensor has detected presence of air in the tubing, the irrigation system may utilize the entire volume of irrigation liquid initially provided in the reservoir by the user, whereby the user may initially provide the same amount of liquid he/she wishes to use during irrigation, without considering internal volumes of the irrigation system as such. Thus, in embodiments, pumping of air into the tubing is allowed for a certain time period without the risk of air being pumped into the user.

In an embodiment, the capacitive sensor comprises an auxiliary processor. The auxiliary processor may be connected to the processor of the control unit. Alternatively, the auxiliary processor may be connected to a transistor (logic gate) electrically coupled to the pump for independent control of the (voltage supply to) the pump, as briefly discussed above. By an auxiliary processor is meant a secondary processor configured to perform certain analyses or calculations specifically related to the capacitive readings of the capacitive sensor. For example, the auxiliary processor may comprise certain filtering mechanisms or means to relate a capacitance reading to a threshold value for the generation of a binary output.

In an embodiment, the auxiliary processor is configured to generate a binary output based on the one or more capacitive characteristics of the fluid in the tubing. For example, the binary output may be sent to the processor of the control unit for further computation. In embodiments, the auxiliary processor is programmable to generate a binary output dependent on a set threshold value. For example, the threshold value may be set according to characteristics of the expected liquids and gasses used with the system, such that a first binary output (e.g., “1”) may be indicative of liquid in the tubing, and the second binary output (then, “0”) may be indicative of gas in the tubing. Thus, in an embodiment, the binary output comprises a first binary output indicative of liquid in the tubing and a second binary output indicative of gas in the tubing. Thereby, the processor of the control unit may interpret this data for the control of the irrigation system, including the control of the pump. Further, as previously discussed, the use of an auxiliary processor configured to generate a binary output may allow for the capacitive sensor to independently control the pump (or another voltage-dependent feature) by means of a transistor (logic gate). Thereby may be provided a system similar to the irrigation system of the first aspect of the invention, but without the need for or provision of a processor of a control unit, but merely comprising an auxiliary processor as disclosed associated specifically with the capacitive sensor and capable of controlling the pump via a transistor/logic gate as disclosed. This may simplify and/or lower costs of an irrigation system. Thus, embodiments relating to the auxiliary processor may be included in such a simplified irrigation system not specifically comprising a processor of a control unit.

In an embodiment, the auxiliary processor comprises a hysteresis function. For example, the hysteresis function may be embodied as an algorithm in the auxiliary processor.

The hysteresis function may reduce fluctuations in the binary output of the auxiliary processor, such as fluctuations as caused by insignificant air bubbles in a stream of liquid passing by the capacitive sensor (e.g., in the tubing). Further, the hysteresis function may filter out minor external disturbances, such as disturbances caused by external objects in the vicinity of the capacitive sensor or irrigation system as such, potentially disturbing the electric field of the capacitive sensor.

In embodiments, the hysteresis function may be embodied by the use of different thresholds dependent on the direction of change of the capacitive characteristic being monitored (e.g., capacitance). For example, if an input signal (e.g., the measured value of capacitance) to the hysteresis function/algorithm is higher than an upper threshold value, the binary output is an upper output value (e.g., “1” indicative of liquid) and if the input signal is lower than a lower threshold value, the binary output is a lower output value (then, “0” indicative of gas), wherein the upper threshold value is greater than the lower threshold value. Thus, between the lower and upper threshold values, the binary output may be maintained according to the most-recent binary output.

In embodiments, the hysteresis function may have a maximum hysteresis of 10%, or of 20%, or of 30%, or of 50%. In embodiments, the hysteresis function may have a maximum hysteresis of at least 10%, or of at least 20%, or of at least 30%, or of at least 40%, or of at least 50%. Generally, stating a maximum hysteresis in percentage may be understood such that, when a standardised target is within X mm of the sensor, the sensor may generate an output, and for the output to change, the target must be moved +Y % of the X mm. For example, where a target within 10 mm of the sensor causes a change in output, the hysteresis function may be adapted to change said output if the object is moved more than 10% away (i.e., in this example, if the target is moved to 11 mm or more). It should be noted that the movement of an object may be an analogy in the present context, and that similar analogies providing the same effect may be provided.

Further, in embodiments, the auxiliary processor may consider both its input at a given time and its past behaviour. For example, the past behaviour may include inputs for the past (trailing) 1 s, or for the past 5 s, or for the past 10 s, or for between 10 s and 60 s, such as up to 60 s. Thereby, at a given time, if the capacitive characteristic was, on average, indicative of liquid in the past (for example) 10 s, a deviation therefrom would not cause a change in the binary output.

The use of a hysteresis function allows for a continuous/uninterrupted operation (pumping) of the irrigation system despite a presence of insignificant air bubbles in a flow of irrigation liquid. For example, insignificant air bubbles may be air bubbles neither affecting the irrigation system nor the user during irrigation, but nonetheless would be detectable by a sensitive capacitive sensor. Thus, the hysteresis function may be designed to, or considered to, purposively lower the sensitivity of the capacitive sensor.

Thereby, the user experiences a smooth irrigation procedure, and the user may not need to pay attention to the presence of insignificant air bubbles in the tubing during an irrigation.

The hysteresis function may relate to, such as constitute or be an embodiment of, the previously discussed embodiment of the processor being configured to terminate (either completely or intermittently/pause) the irrigation procedure of the reservoir in response to the characteristics of the fluid in the tubing being indicative of air for a time period exceeding a first threshold.

In an alternative embodiment, or in addition, the processor of the control unit may comprise a hysteresis function having the same features and/or providing the same benefits as disclosed above.

In an embodiment, the capacitive sensor comprises a capacitive proximity sensor. By a capacitive proximity sensor is meant a sensor type suited to detect presence of nearby objects/materials without any physical contact. In particular, a capacitive proximity sensor utilizes the change of capacitance based on a change in the electrical field around an active face (in the following also denoted a sensor plate) of the sensor. The target/object to be sensed will act as the second plate of a plate capacitor thus formed between the active face of the sensor and said target. In other words, a capacitive proximity sensor may thus work without a second fixated plate and yet be modelled according to a plate capacitor, as the target to be sensed will act as the second plate. Typically, the proximity sensor comprises an internal oscillator circuit such that, as the target approaches the active face, oscillations increase until they reach a threshold level and activate/generate an output, such as a binary output as previously discussed. The threshold level may be adjusted according to the desired use of the proximity sensor.

A proximity sensor may also be denoted a touch sensor or a distance sensor in the field.

The capacitive proximity sensor may be a 6-pin single-channel proximity detector. The sensor may have an operative voltage in the range from 2.0 V to 5.5 V.

The auxiliary processor as previously disclosed may form part of the capacitive proximity sensor. The capacitive proximity sensor may comprise, as part of its auxiliary processor, the hysteresis function as previously disclosed.

In embodiments, the proximity sensor is configured to measure the capacitance as induced by a medium (e.g., fluid) in proximity to said proximity sensor. Correspondingly, in an alternative wording, the proximity sensor may be configured to measure the permittivity of the medium (e.g., fluid) in proximity to the proximity sensor. More specifically, and in accordance with embodiments previously disclosed, in embodiments, the proximity sensor is configured to measure characteristics of a (dielectric) medium in the tubing arranged between the sensor plate and the ground plate, said characteristics of the medium including the permittivity of said medium.

In an embodiment, the capacitive proximity sensor comprises a sensor plate arranged adjacent to the tubing. The sensor plate may also be denoted an active face of the sensor. The sensor plate may thus be considered the first plate of a plate capacitor as previously discussed. The sensor plate may be facing and arranged adjacent to the tubing, such as abutting the tubing. For example, where the sensor plate is substantially planar and the tubing is substantially circular (in cross section), the sensor plate may be arranged parallel to a tangent plane of an outer surface of the tubing. In an alternative embodiment, the sensor plate may be curved or adapted/fitted to an outer surface of the tubing, such as to provide a constant radial distance to a centre point of the tubing (e.g., where the tubing is substantially circular in cross section), such as to provide a stable signal less influenced by other objects than the contents (fluids) in the tubing.

The sensor plate may have a length, in the direction parallel with the extension of the tubing, between 1 mm and 30 mm, such as between 1 mm and 20 mm, or between 1 mm and 10 mm. In embodiments, the sensor plate has a length of 3 mm in the direction parallel with the extension of the tubing.

The sensor plate may have a width, in the direction perpendicular with the extension of the tubing, of between 5 mm and 40 mm, such as between 5 mm and 20 mm, such as between 10 mm and 20 mm.

In an embodiment, the capacitive proximity sensor further comprises an electrically grounded ground plate arranged adjacent to the tubing and opposite to the sensor plate to form a capacitor wherein the tubing is arranged between the sensor plate and the ground plate.

By an electrically grounded ground plate is meant a plate electrically insulated from the sensor plate and electrically grounded relative to said sensor plate. For example, the ground may be a floating ground, such as grounded to the power source powering the capacitive sensor.

In embodiments, the sensor plate and the ground plate are fixated relative to each other and have a defined geometry. Thereby, the areas A and their separation distance d are defined, whereby the capacitance C is proportional to the permittivity E of the medium/fluid between the plates. In embodiments where the sensor and ground plates are not parallel, the separation distance may be considered an effective separation distance.

The ground plate may be arranged adjacent to (such as abutting) the tubing and opposite to the sensor plate. By being positioned opposite to the sensor plate is meant that the sensor plate and the ground plate faces each other to form a (plate) capacitor, wherein the tubing is thus arranged between the sensor plate and the ground plate. For example, when arranged in relation to the tubing, the sensor plate and the ground plate may each be arranged in parallel with, such as abutting, the tubing extending between said plates. In other words, the sensor plate may be arranged in parallel with a first tangent plane to an outer surface of the tubing and the ground plate may be arranged in parallel with a second tangent plane to the outer surface of the tubing, wherein the second tangent plane is arranged diametrically opposite the first tangent plane, such that the first tangent plane and the second tangent plane are parallel.

Thereby, the ground plate may form the opposite plate of a plate capacitor, wherein the plate capacitor thus comprises the sensor plate and the ground plate, which are separated by means of the ground plate being arranged adjacent to the tubing and opposite to the sensor plate.

The ground plate may have dimensions substantially similar to those of the sensor plate, or the dimensions may differ.

The ground plate may have a length, in the direction parallel with the extension of the tubing, between 1 mm and 30 mm, such as between 1 mm and 20 mm, or between 1 mm and 10 mm. In embodiments, the sensor plate has a length of 3 mm in the direction parallel with the extension of the tubing.

The ground plate may have a width, in the direction perpendicular with the extension of the tubing, of between 5 mm and 40 mm, such as between 5 mm and 20 mm, such as between 10 mm and 20 mm.

In an embodiment, the sensor plate and/or the ground plate are fitted according to an outer surface geometry of the tubing. Thereby, a constant radial distance from the sensor plate or ground plate to a centre point of the tubing (e.g., where the tubing is substantially circular in cross section) may be provided, such as to provide a stable signalless influenced by other objects in the vicinity of the electric field of the capacitor. For example, where the tubing is substantially circular in cross section, the sensor plate and/or the ground plate may each be adapted/fitted to the curvature of the outer surface of the tubing and extend along said tubing for up 180° about the tubing (e.g., about a centre point of the tubing). The sensor plate and the ground plate may not both be fitted according to the outer surface geometry of the tubing. For example, in embodiments, only the sensor plate is fitted according to the outer surface geometry of the tubing, whereas the ground plate is a planar plate.

In a second aspect of the invention, a method of controlling an irrigation procedure in, or a pump or valve of, an irrigation system according to the first aspect of the invention is disclosed. The method is performed by a processor of the irrigation system, such as the processor of the control unit or the auxiliary processor of the capacitive sensor (e.g., where the pump is controlled by a binary output of the auxiliary processor without influence from a processor of the control unit). The reservoir of the irrigation system comprises an irrigation liquid. For example, the user has prepared the system by means of pouring an irrigation liquid into the reservoir prior to initiating the irrigation procedure.

The irrigation system is turned on, such that the irrigation procedure may be initiated according to the method. The method may comprise the steps of:
initiating the irrigation procedure;
measuring one or more capacitive characteristics of a fluid in the tubing; and
controlling the irrigation procedure (e.g., controlling the pump) in accordance with the one or more capacitive characteristics of the fluid in the tubing.

The irrigation procedure may be initiated in response to a user interacting with a user interface, e.g., an on/off button, or the processor may instruct the pump to start. It is appreciated that the irrigation procedure may be performed even without association with a human body. In particular, it is appreciated that “initiating the irrigation procedure” may merely be a reference to allow liquid to flow through the tubing, and “controlling the irrigation procedure” may be embodied by controlling the pump, a valve, or another element or feature of the irrigation system.

The step of measuring one or more capacitive characteristics may comprise receiving, by the processor of the control unit, data indicative of the one or more capacitive characteristics from the capacitive sensor.

It is appreciated that embodiments and related effects and/or benefits of the first aspect of the invention may be considered applicable to the method according to the second aspect of the invention. For example, where embodiments of the first aspect of the invention disclose or entail steps of a method, it is appreciated that such steps of a method may be applicable to the method of the second aspect of the invention.

In an embodiment, controlling the irrigation procedure comprises terminating (either completely or intermittently/pause) the irrigation procedure in accordance with the one or more capacitive characteristics of the fluid in the tubing being indicative of gas. Controlling the irrigation procedure may comprise controlling the pump, such that, in embodiments, controlling the irrigation procedure comprises terminating (either completely or intermittently/pause) pumping in accordance with the one or more capacitive characteristics of the fluid in the tubing being indicative of gas

Also disclosed is a method of operating an irrigation system according to the first aspect of the invention, the method comprising the steps of:
arranging an irrigation liquid in the reservoir;
initiating pumping of liquid, thereby facilitating a flow of irrigation liquid from the reservoir, through the tubing, to a catheter coupled to the tubing;
measuring the capacitive characteristics of a fluid in the tubing; and
controlling the irrigation procedure (e.g., controlling the pump) in accordance with the capacitive characteristics of the fluid in the tubing.

The method of operating an irrigation system may be performed by a user and one or more processors of the irrigation system in unison.

Also disclosed is an alternative irrigation system configured to run an irrigation procedure for irrigation of the bowels of a user, the system comprising:
a housing comprising a reservoir for containing an irrigation liquid;
a tubing providing fluid communication between the reservoir and a catheter for use with the irrigation system;
a pump for facilitating a flow of irrigation liquid from the reservoir, through the tubing, to the catheter, the pump being connected to a power source;
a capacitive sensor configured to measure one or more capacitive characteristics of a fluid in the tubing; and
a logic gate (transistor) arranged in series between the power source and the pump, and connected to the capacitive sensor;

wherein the capacitive sensor (such as an auxiliary processor thereof) is configured to generate a binary output, and wherein the logic gate is configured to terminate power to the pump in accordance with receiving a binary input from the capacitive sensor being indicative of the one or more capacitive characteristics of a fluid in the tubing being indicate of gas.

The alternative irrigation system reflects the previously discussed embodiments wherein the capacitive sensor alone, such as by means of an auxiliary processor associated with the capacitive sensor, may control the irrigation procedure through the provision of a logic gate/transistor. In a preferred embodiment, the gate is an AND-gate.

The alternative irrigation system may comprise one or more features disclosed in relation to the first aspect of the invention, and as such may be combined with embodiments thereof to provide a simple irrigation system not comprising a control unit comprising a processor, but merely the auxiliary processor disclosed in relation to the capacitive sensor of the first aspect of the invention. The alternative irrigation system may comprise means for powering on the system without the influence of a processor. For example, the alternative irrigation system may be powered on by a switch.

It is appreciated that embodiments and related effects and/or benefits of the first aspect of the invention may be applicable to the alternative irrigation system as disclosed herein. For example, where embodiments of the first aspect of the invention disclose features not immediately dependent on the processor of the control unit, it is appreciated that such features may be applicable to the alternative irrigation system as disclosed herein. In addition to the benefits disclosed above in relation to the first aspect of the invention, the alternative irrigation system as disclosed herein provides for a simple irrigation system being cost-efficient yet provides a similar, albeit simplified, mechanism to respond to a change of fluid in the tubing.

### DETAILED DESCRIPTION OF THE DRAWINGS

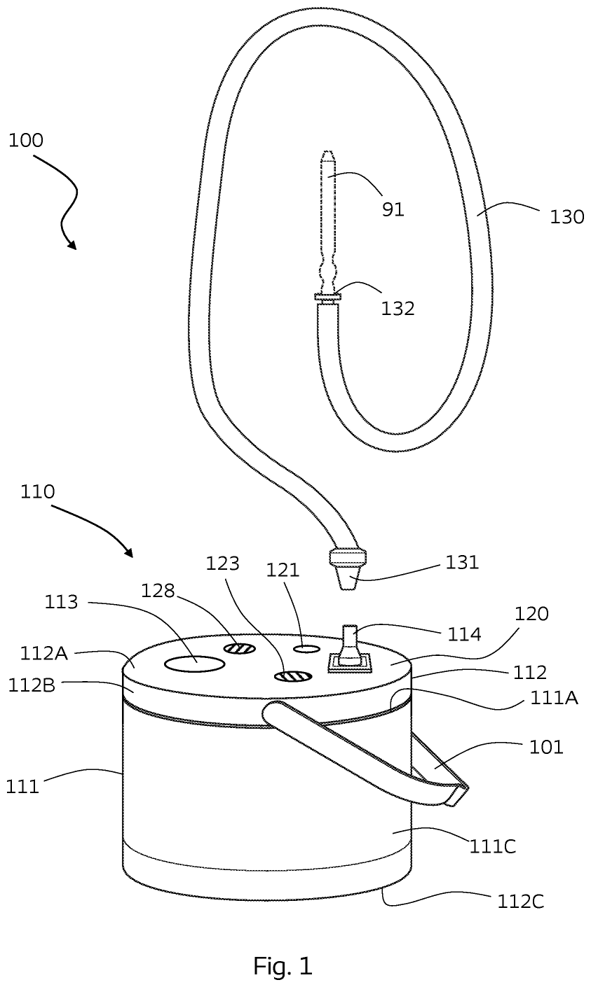

FIG. 1 illustrates an exemplary irrigation system 100 according to an embodiment of the invention. The irrigation system 100 comprises a housing 110 comprising a reservoir 111 and an electronics compartment 120 sealed from the reservoir 111. The reservoir 111 is closed off with a lid 112. In the illustrated embodiment, the electronics compartment 120 is contained in the lid 112.

The lid 112 may be detachable from the reservoir 111 to allow for filling the reservoir with an irrigation liquid and/or for easy cleaning of the interior of the reservoir. In addition, or alternatively, the lid 112 may comprise an auxiliary opening 113 providing access to the reservoir 111 through the lid. The auxiliary opening 113 may be sealable by an auxiliary lid associated with the auxiliary opening. The lid 112 is adapted to hermetically close off the reservoir 111.

The reservoir 111 may comprise a preferred bottom 111C. The preferred bottom 111C may be arranged opposite the lid 112 arranged in a top portion 111A of the reservoir 111. A wall portion 111B may extend from the preferred bottom 111C to the top portion 111A. Thus, the bottom 111C, the wall portion 111B and the top portion 111A may be said to define the reservoir 111. The preferred bottom 111C may comprise a plane surface such that the user may arrange the reservoir 111 on a table, floor or similar during irrigation on said preferred bottom.

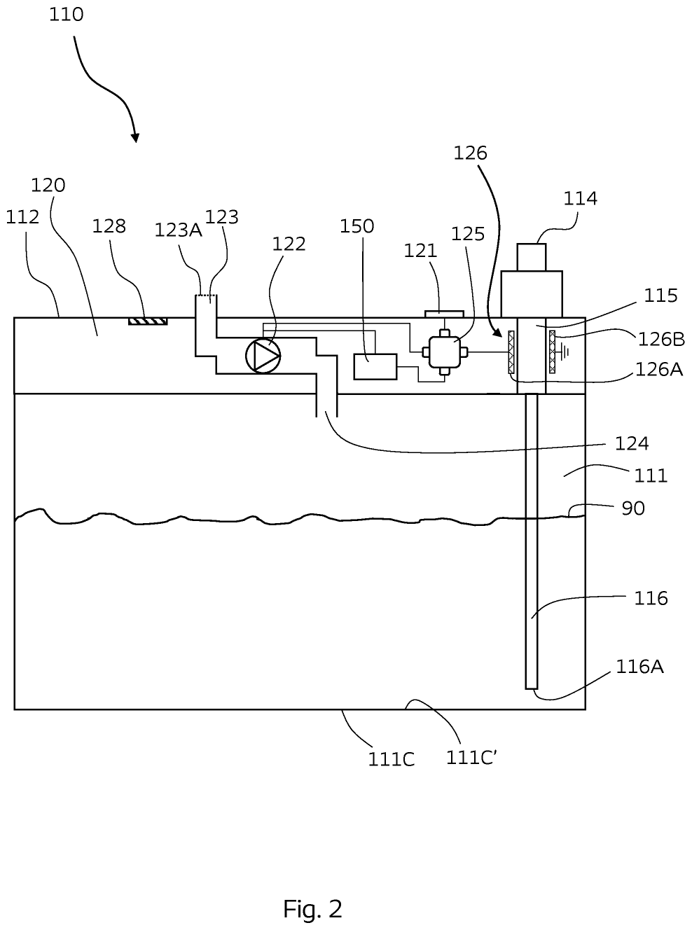

The irrigation system 100 further comprises a flexible tubing 130 providing fluid communication between an interior of the reservoir 111 and a catheter 90 for use with the irrigation system (see also FIG. 2 for a detailed view of the interior of the reservoir 111). The flexible tubing 130 may be detachable from the housing 110 via a first connector 131 configured to mate with an associated tube connector 114 of the lid 112. The flexible tubing 130 comprises a second connector 132 arranged in an opposite end of the tubing relative to the first connector 131. The second connector 132 is configured to mate with an associated connector of a catheter 91 for use with the irrigation system 100.

The irrigation system 100 may comprise a handle 101 for easy carrying. The handle 101 may be attached to the lid 113, or the handle 101 may be attached to the reservoir 111.

The irrigation system 100 may comprise a control unit comprising a processor. In the embodiment, the control unit is built into the lid 112. The control unit may comprise an interface 121, such as a user interface for providing information to the user about the operating status of the system. For example, the interface 121 may be a light diode/LED, a graphical user interface/screen, or means for providing an audible or haptic feedback. The interface 121 may be configured to receive user input. For example, the interface 121 may be a button configured to turn the system on/off. The system may comprise one or more user interfaces 121.

The irrigation system 100 comprises an air intake 122. The air intake 123 may be arranged in the lid 112, such as in a top portion 112A or in a rim portion 112B of the lid. The air intake 123 provides ambient air for an air pump arranged in the system 100, such as in the electronics compartment 120. The pump is discussed in greater detail in relation to FIG. 2.

The electronics compartment 120 may be vented to the surroundings by means of a vent 128.

FIG. 2 illustrates an exemplary cross-sectional view of the housing 110 of an irrigation system 100 according to an embodiment of the invention. The housing 110 comprises the reservoir 111, wherein an irrigation liquid 90 is indicated, and the electronics compartment 120. In the illustrated embodiment, the electronics compartment 120 is arranged in the lid 112. The electronics compartment 120 contains the majority of electronics associated with the irrigation system 110 including the pump 122, the at least one processor 125 and the capacitive sensor 126 arranged to measure one or more capacitive characteristics of the tubing, and in particular of the intermediate tube section 115. The capacitive sensor 126 is connected to the processor 125 in the illustrated embodiment.

The tube connector 114 of the lid 112 is in fluid communication with the reservoir 111 via the intermediate tube section 115 provided in the lid and a dip tube 116 extending into the reservoir. The dip tube 116 comprises an opening 116A wherein liquid may enter the dip tube and flow further through the tubing of the system 100. The dip tube 116 extends into close proximity of an internal bottom 111C′ of the reservoir 111 such that for a majority of the irrigation procedure, the dip tube 116 is covered with irrigation liquid 90, when the preferred bottom 111C of the reservoir 111 is arranged on a plane surface. Thereby, since the irrigation system 100 is based on an air pump 122 configured to pressurize the (air of the) reservoir 111 to facilitate a flow of irrigation liquid 90 through the tubing 130, said liquid 90 may enter the dip tube 116 via the opening 116A until said opening is not covered with irrigation liquid 90. Thus, in a preferred embodiment, the opening 116A of the dip tube 116 is arranged in close proximity of the internal bottom 111C′, or the opening 116A may be arranged within the internal bottom 111C′ via adequate arrangement of the dip tube 116.

The electronics compartment 120 may be vented relative to the surroundings via the vent 128.

The air pump 122 comprises an air intake 123. Preferably, the air intake 123 comprises a filter 123A. The air pump 122 comprises an air outlet 124 in communication with the interior of the reservoir 111. Thereby, the air pump 122 may pressurize the reservoir 111 by taking in ambient air via the air intake 123 and outputting the air via the air outlet 124. The pump 122 is connected to the at least one processor 125 such that the at least one processor may control the pump, such as in accordance with the one or more capacitive characteristics as measured by the capacitive sensor 126.

The irrigation system 100 comprises a power unit 150 coupled to the processor 125 and the pump 122. Preferably, the power unit 150 is arranged in the electronics compartment 125. The power unit 150 provides operating voltage to the processor and the pump. The power unit can be a battery, such as a rechargeable battery. The irrigation system may further comprise further electronic features not illustrated, such as means for generating, transmitting and receiving a wireless signal, a memory coupled to the processor and further sensors.

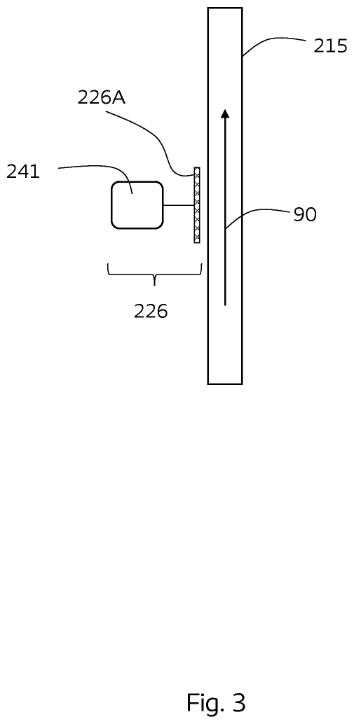

FIG. 3 illustrates a capacitive sensor 226 according to an embodiment of the invention. The capacitive sensor 226 is arranged relative to a tubing 215, such as the intermediate tube section 115 or the dip tube 116 of FIG. 2 or the flexible tubing 130 of FIG. 1. In a preferred embodiment, the capacitive sensor 226 is arranged in the electronics compartment 120 of FIG. 2, such that the capacitive sensor 226 is arranged relative to the intermediate tube section 115.

The capacitive sensor 226 of FIG. 2 is a capacitive proximity sensor comprising an auxiliary processor 241 connected to a sensor plate 226A. The sensor plate 226A is arranged adjacent to the tubing 215, such as abutting the tubing 215. The tubing 215 may be circular in cross-section, and the sensor plate 226A may be planar and parallel with a tangent plane of the tubing. Alternatively, the sensor plate 226A may be adapted or fitted to the curvature of the tubing 215

In a preferred embodiment, the auxiliary processor 241 is connected to the processor 125 of FIG. 2. In an alternative embodiment, the auxiliary processor 241 is connected to a logic gate arranged in series between a power source and the pump, such that the logic gate may, in accordance with receiving a binary input from the auxiliary processor, terminate power to the pump.

The capacitive sensor, such as the auxiliary processor thereof, is configured to measure one or more capacitive characteristics of a fluid 90 flowing through the tubing 215. For example, the one or more capacitive characteristics may be capacitance. The auxiliary processor 241 may be configured to generate a binary output based on whether the capacitance is above or below a threshold value indicative of whether the fluid is liquid or gas. Thereby, the processor 125, or the logic gate in the alternative embodiment, may receive such binary input from the auxiliary processor 241 and control the irrigation procedure in accordance wherewith.

In the illustrated embodiment, the capacitive sensor 226 solely comprises a sensor plate 226A. In such an embodiment, the fluid passing by such sensor plate 226A will cause capacitive alterations of the electric field and thereby constitute a signal. Such a capacitive sensor 226 may be particularly simple and easy to implement.

The sensor plate 226A may have a length, in the direction parallel with the extension of the tubing 215, between 1 mm and 30 mm, such as between 1 mm and 20 mm, or between 1 mm and 10 mm. In embodiments, the sensor plate has a length of 3 mm in the direction parallel with the extension of the tubing.

The sensor plate 226A may have a width, in the direction perpendicular with the extension of the tubing, of between 5 mm and 40 mm, such as between 5 mm and 20 mm, such as between 10 mm and 20 mm.

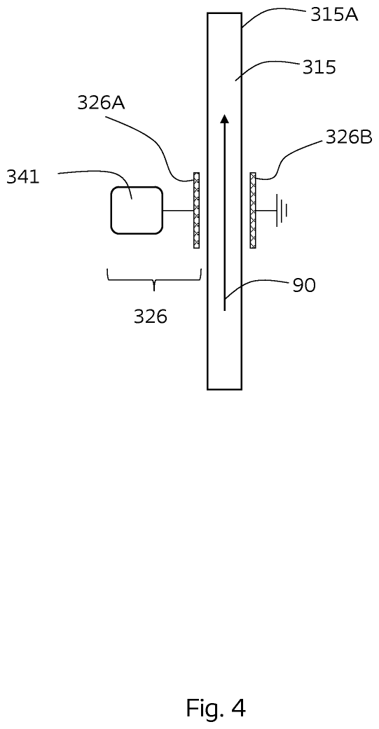

FIG. 4 illustrates a capacitive sensor 326 according to an embodiment of the invention. The capacitive sensor 326 is arranged relative to a tubing 315, such as the intermediate tube section 115 or the dip tube 116 of FIG. 2 or the flexible tubing 130 of FIG. 1. In a preferred embodiment, the capacitive sensor 326 is arranged in the electronics compartment 120 of FIG. 2, such that the capacitive sensor 326 is arranged relative to the intermediate tube section 115.

The build of the capacitive sensor 326 is similar to the build of the capacitive sensor 226 of FIG. 3, but with the addition of an electrically grounded ground plate 326B arranged adjacent to the tubing 315 and opposite to the sensor plate 326A to form a capacitor wherein the tubing 315 is arranged between the sensor plate 326A and the ground plate 326B.

The ground plate 326B is electrically grounded relative to the sensor plate 326A. For example, the ground may be a floating ground, such as grounded to a power supply/battery powering the capacitive sensor 326.

The provision of a ground plate 326B may reduce external noise caused, for example, by objects in vicinity of the capacitive sensor 326, which could potentially disturb the electrical field generated by the sensor plate 326A, or the sensor plate 226A of FIG. 3.

The sensor plate 326A and the ground plate 326B are arranged adjacent to the tubing 315. The tubing 315 may be circular in cross-section, and the sensor plate 326A and the ground plate 326B may be planar and parallel with a tangent plane of the tubing. The ground plate 326B is arranged opposite to the sensor plate 326A. By being positioned opposite to the sensor plate 326A is meant that the sensor plate 326A and the ground plate 326B faces each other to form a (plate) capacitor. For example, when arranged in relation to the tubing 315, the sensor plate 326A and the ground plate 326B may each be arranged in parallel with, such as abutting, the tubing 315 extending between said plates 326A,326B. In other words, the sensor plate 326A may be arranged in parallel with a first tangent plane to an outer surface 315A of the tubing 315 and the ground plate 326B may be arranged in parallel with a second tangent plane to the outer surface 315A of the tubing 315, wherein the second tangent plane is arranged diametrically opposite the first tangent plane, such that the first tangent plane and the second tangent plane are parallel. Alternatively, the sensor plate 326A and/or the ground plate 326B may be adapted or fitted to the curvature of the tubing 315.

The ground plate 326B may have dimensions substantially similar to those of the sensor plate 326A, or the dimensions may differ.

The ground plate 326B may have a length, in the direction parallel with the extension of the tubing 315, between 1 mm and 30 mm, such as between 1 mm and 20 mm, or between 1 mm and 10 mm. In embodiments, the sensor plate has a length of 3 mm in the direction parallel with the extension of the tubing.

The ground plate 326B may have a width, in the direction perpendicular with the extension of the tubing, of between 5 mm and 40 mm, such as between 5 mm and 20 mm, such as between 10 mm and 20 mm.

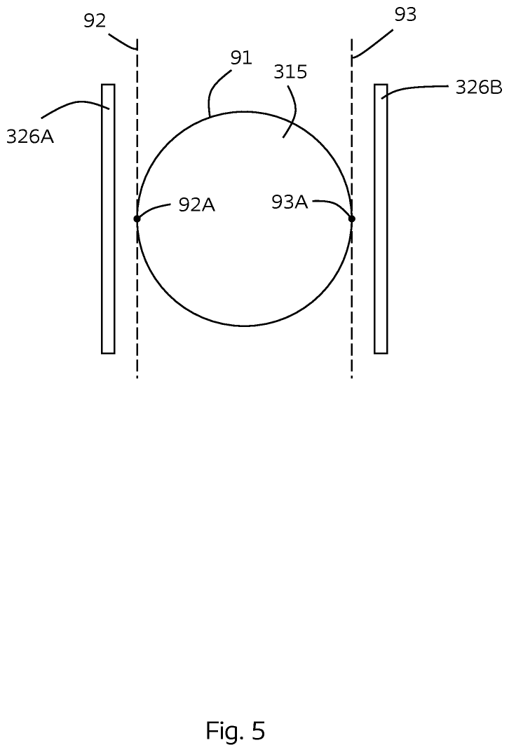

FIG. 5 illustrates a circle 91 and two tangent planes 92,93 (dashed) thereof being mutually parallel. The illustration serves to illustrate how the sensor plate 326A and ground plate 326B of FIG. 4 may be arranged relative to each other. Namely, where the circle 91 is the cross-sectional view of a tubing, such as the tubing 315 of FIG. 4, the sensor plate 326A may be arranged in parallel with a first tangent plane 92, where the first tangent plane is a tangent plane to a first point 92A on the circle 91 (corresponding to an outer surface of the tubing). Likewise, the ground plate 326B may be arranged in parallel with a second tangent plane 93, where the second tangent plane is a tangent plane to a second point 93A on the circle 91, arranged diametrically opposite the first point 92A, such that the first tangent plate 92 and the second tangent plane 93 are parallel. Thereby, correspondingly, the sensor plate 326A and the ground plate 326B are likewise arranged adjacent to the tubing and opposite each other such that the tubing 315 is arranged between the sensor plate 326A and the ground plate 326B.

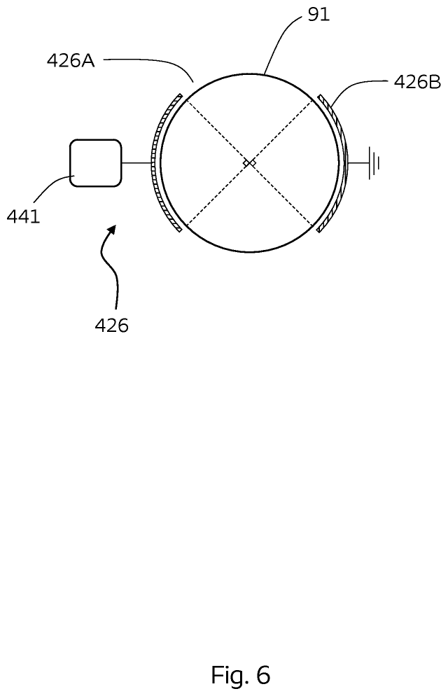

FIG. 6 illustrates an exemplary capacitive sensor 426 according to an embodiment of the invention. The capacitive sensor 426 comprises a sensor plate 426A and a ground plate 426B fitted according to the curvature of the tubing 415. The sensor plate 426A is connected to an auxiliary processor 441 as previously discussed. The ground plate 426B is electrically grounded as previously discussed.

In the present embodiment, the cross-section of the tubing 415 is circular, meaning that the curvature of the tubing is circular or, when considering the direction perpendicular to the illustration, cylindrical. The sensor plate 426A and ground plate 426B are adapted or fitted according to this shape, such that each of the sensor plate 426A and ground plate 426B resembles semicircles arranged concentrically about the tubing 415. In the illustrated embodiment, each plate 426A,426B spans an angle of 90 degrees as illustrated by dashed radii and are arranged opposite each other, such that they are separated by a like angle of 90 degrees. In alternative embodiments, the plates 426A,426B each spans an angle between 45 degrees and 180, preferably between 45 and 170 degrees, such as between 67.5 and 157.5 degrees, or between 90 degrees and 135 degrees. Thereby, an effective separation between the plates is increased and electric field contributions caused by end-portions of each curved plate being in closer proximity than the rest of the plates are reduced.

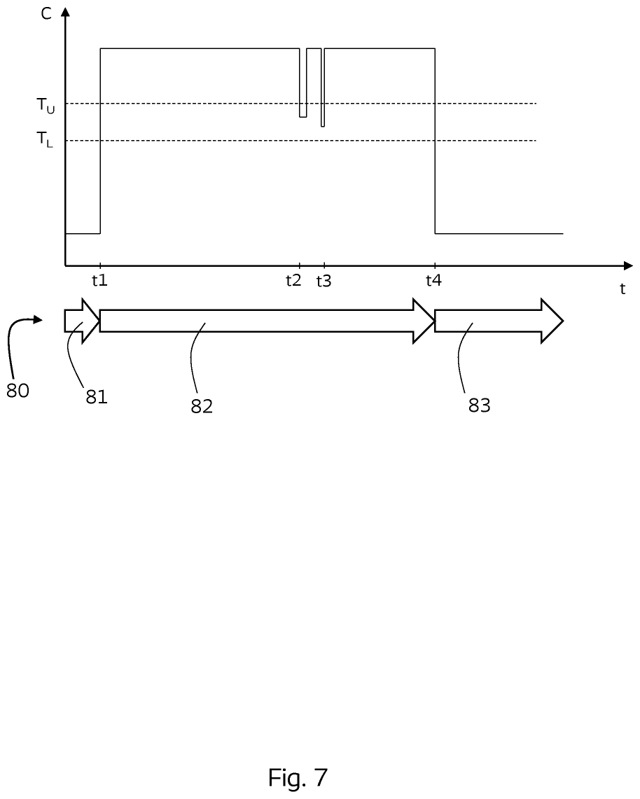

FIG. 7 illustrates an exemplary graph depicting the capacitance as a function of time t. A graph depicting the permittivity as a function of time would look identical when the dimensions and arrangement of the capacitor are unchanged over time due to the relation between capacitance, permittivity and physical dimensions of the capacitor. Thus, the graph serves to illustrate how the capacitance—or permittivity—changes as a function of time for a capacitor when a fluid passes through the capacitor (e.g., for the case of a (parallel) plate capacitor). In the present context, the fluid can be either a liquid or a gas, and the fluid may flow through a tubing arranged within or in proximity to the capacitor as illustrated in previous figures.

Thus, the graph depicts, in an exemplary manner, the output from a capacitive sensor according to embodiments of the invention. In particular, the graph depicts the time-dependent relationship for a parallel-plate capacitor according to embodiments:

$$C ( t ) = ε ( t ) \frac{A}{d} = {ε}_{r} ( t ) {ε}_{0} \frac{A}{d}$$

Initially, the capacitance C(t) (and, correspondingly, ε(t)) is low, indicating that the permittivity of the medium in the capacitor is low (e.g., air).

At time t1, the capacitance increases, indicating that the permittivity of the medium in the capacitor is now high, thus indicating the presence of a different fluid having a different (relative) permittivity: air has been replaced with liquid in the tubing—indicating that pumping of irrigation liquid has started and thus, that liquid is passing through the tubing arranged in, or in proximity to, the capacitive sensor.

At time t2, the capacitance starts fluctuating to indicate intermittent presence of a low-permittivity medium (e.g., air) until time t3. Thus, the period between t2 and t3 illustrates minor fluctuations in the capacitance, which may be indicative of air bubbles passing through the tubing, and thus by the capacitor. According to embodiments of the invention, the capacitive sensor, such as an auxiliary processor thereof, comprises a hysteresis function designed to disregard air bubbles, if such air bubbles do not cause the capacitance to fall below the lower threshold T_{L}.

In other words, the minor fluctuations between time t3 and t4, potentially caused by air bubbles, do not cause the capacitance to decrease below a lower threshold T_{L}, and as such, no output being indicative of air is generated. For example, the air bubbles may be insignificant, small, and/or pass by the capacitor by such a speed that the capacitance does not drop below the lower threshold T_{L}. An upper threshold T_{U} is indicated as well.

Through the provision of a hysteresis function designed to disregard insignificant air bubbles, the user may experience a smooth/undisturbed irrigation procedure irrespective of the presence of insignificant air bubbles. Further, the system may still be able to terminate the irrigation procedure in case significant amount of air is starting to be pumped through the tubing (e.g., after time t4).

At time t4, the capacitance decreases, indicative of air, and remains low. Thus, the low capacitance following time t4 may be indicative of the reservoir being emptied of irrigation liquid.

According to embodiments of the present invention, the processor of the control unit is configured to control the irrigation procedure in accordance with the capacitive characteristics of the fluid in the tubing. For example, the processor may terminate, e.g., pause, the irrigation procedure at time t4 such that air is not pumped through the tubing.

FIG. 7 further comprises a timeline 80 aligned with the events at time t1 and t4 of the exemplary graph. The timeline 80 illustrates the status of the irrigation procedure (e.g., the timeline illustrates the status of the pump). Initially, during a first time block 81 the irrigation procedure is off, e.g., the pump is off, whereby no liquid is being pumped through the tubing. At time t1, the irrigation procedure is turned on (e.g., the pump is turned on), whereby liquid enters the tubing. Thus, the irrigation procedure is on during the second time block 82. At time t4, the capacitive characteristics become indicative of air in the tubing (and fall below the lower threshold T_{L}), which in turn may be indicative of the reservoir being empty. In response to the presence of air in the tubing, the irrigation procedure is turned off. Thus, the third time block 83 illustrates the irrigation procedure being off (e.g., the pump is turned off).

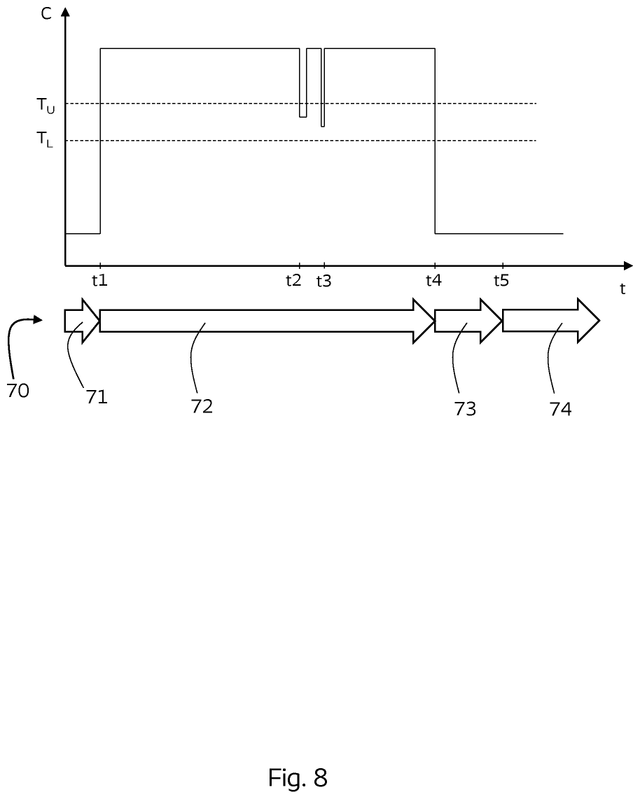

FIG. 8 illustrates the exemplary graph of FIG. 7, wherein a timeline 70 aligned with events at time t1, t4 and t5 is included. Contrary to the embodiment of FIG. 7, the present embodiment includes an event at time t5. The first time block 71 corresponds to the first time block 81 of FIG. 7 and the second time block 72 corresponds to the second time block 82 of FIG. 7.

Following the determination of gas in the tubing at time t4, the pump may stay on in the third time block 73 until time t5, whereafter the pump is turned off: in the fourth time block 74, the pump is off.

The figure serves to illustrate that the processor may be configured to terminate the irrigation procedure in response to the characteristics of the fluid in the tubing being indicative of air for a time period exceeding a first threshold. Thus, the processor may be configured to terminate the irrigation procedure in response to the characteristics of the fluid in the tubing being indicative of air after X seconds (i.e., the length of the third time block 73) after first measuring (i.e., at time t4) a capacitive characteristic being indicative of air, where the X seconds may be selected between 10 s and 60 s.

For example, if the one or more capacitive characteristics of the fluid in the tubing are indicative of air for a certain time period (the third time block 73) (e.g., following a period (the second time block 72) of the characteristics of the fluid in the tubing being indicative of liquid, meaning that the irrigation procedure is ongoing), the processor may be configured to terminate (at time t5) the irrigation procedure, such as to turn off the pump and/or close a valve. For example, air may become present in the tubing once the reservoir is emptied of irrigation liquid. However, due to an internal volume of the tubing, pressurization/pumping of liquid may continue for a certain period of time (length of the third time block 73) during which said internal volume of the tubing is emptied of liquid. For example, the first threshold may be set according to an internal volume of the tubing and/or a pumping speed of the irrigation system.

By allowing air to be present in the tubing for a certain time period after the capacitive sensor has detected presence of air in the tubing (i.e., the third time block 73), the irrigation system may utilize the entire volume of irrigation liquid initially provided in the reservoir by the user, whereby the user may initially provide the same amount of liquid he/she wishes to use during irrigation, without considering internal volumes of the irrigation system as such. Thus, in embodiments, pumping of air into the tubing is allowed for a certain time period 73 without the risk of air being pumped into the user.

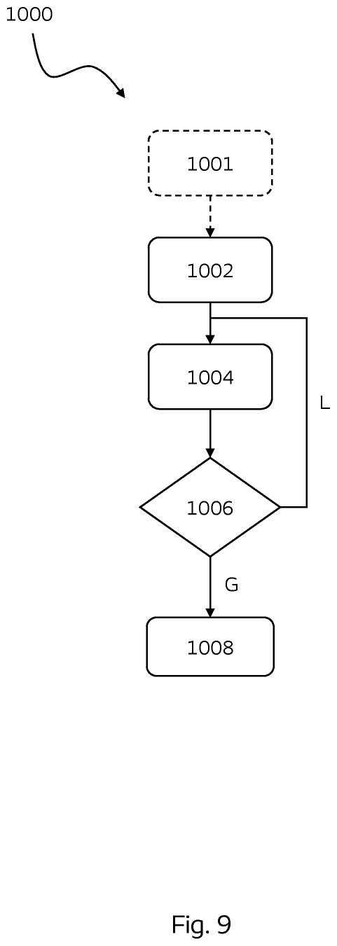

FIG. 9 illustrates a method 1000 of controlling an irrigation procedure in an irrigation system according to embodiments of the invention. The method 1000 is performed by a processor of the irrigation system, such as the processor 125 of FIG. 2.

The reservoir of the irrigation system comprises an irrigation liquid. For example, prior to execution of the method 1000, a user has prepared the irrigation system by providing the irrigation liquid in the reservoir.

The method 1000 comprises the steps of:
initiating 1002 the irrigation procedure;
measuring 1004 one or more capacitive characteristics of a fluid in the tubing; and
controlling 1006 the irrigation procedure in accordance with the one or more capacitive characteristics of the fluid in the tubing.

The method 1000 may comprise the optional (dashed box) initial step of turning on 1001 the pump. For example, the processor may turn on the pump in response to receiving a user input indicative of turning on the pump. Alternatively, the pump may be turned on manually by means of a switch actuated by a user.

The step of initiating 1002 the irrigation procedure may comprise, where the pump is an air pump, to initiate pressurization of the reservoir such that liquid starts flowing through the tubing. In embodiments, the step of initiating the irrigation procedure may be contained in the step of turning on 1001 the pump in cases where such turning on of the pump results in immediate pressurization.

The step of controlling 1006 the irrigation procedure may comprise to continuously measure the one or more capacitive characteristics and send appropriate instructions to the pump. For example, where the one or more capacitive characteristics are indicative of liquid L, the processor may do nothing, such that the pump continues to operate, and such that the method continues to measure 1004 one or more capacitive characteristics of the fluid in the tubing.

The step of controlling 1006 the irrigation procedure may comprise the step of terminating 1008 the irrigation procedure in accordance with the one or more capacitive characteristics of the fluid in the tubing being indicative of gas G.

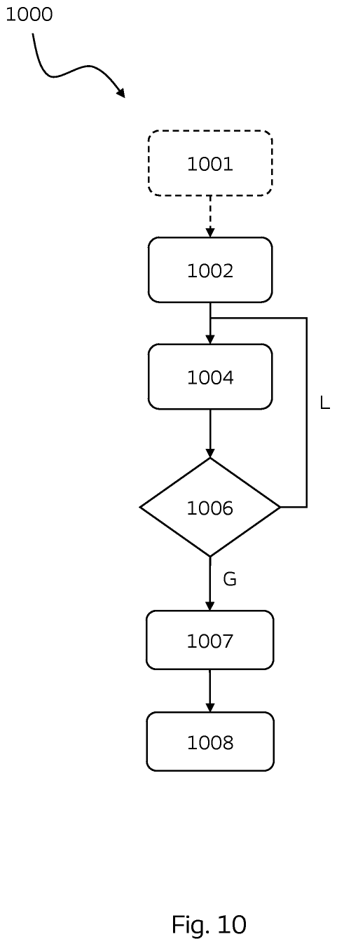

FIG. 10 illustrates the method 1000 of controlling an irrigation procedure as discussed in relation to FIG. 9, but with an intermediate step, inserted in response to determining that gas G is present in the tubing and prior to the step of terminating 1008 the irrigation procedure, of waiting 1007 for a set period of time before terminating 1008 the irrigation procedure. During such step of waiting 1007 for a set period of time, the irrigation procedure remains on (pumping is on), such that gas is pumped through the tubing. Thereby, when the period of time is set according to an internal volume of the tubing and a pumping speed of the pump, the tubing may be emptied of liquid, without gas eventually being pumped into the rectum of the user.

The use of the terms “first”, “second”, “third” and “fourth”, “primary”, “secondary”, “tertiary” etc. does not imply any particular order but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering.

Furthermore, the labelling of a first element does not imply the presence of a second element and vice versa.

It is to be noted that the words “comprising” and “including” do not necessarily exclude the presence of other elements or steps than those listed.

It is to be noted that the words “a” or “an” preceding an element or method step do not exclude the presence of a plurality of such elements or method steps.

It should further be noted that any reference signs do not limit the scope of the claims, that the exemplary embodiments may be implemented at least in part by means of both hardware and software, and that several “means”, “units” or “devices” may be represented by the same item of hardware.

The various exemplary methods, devices, and systems described herein are described in the general context of method steps processes or actions by a system or processor, which may be implemented in one aspect by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that perform specified tasks or implement specific abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

Although particular features have been shown and described, it will be understood that they are not intended to limit the claimed invention, and it will be made obvious to those skilled in the art that various changes and modifications may be made without departing from the spirit and scope of the claimed invention. Accordingly, the specification and drawings are to be regarded in an illustrative rather than restrictive sense. The claimed invention is intended to cover all alternatives, modifications, and equivalents.

A list of embodiments is set out in the following items:
1. An irrigation system configured to run an irrigation procedure for irrigation of the bowels of a user, the system comprising:
   a housing comprising a reservoir for containing an irrigation liquid;
   a tubing providing fluid communication between the reservoir and a catheter for use with the irrigation system;
   a pump for facilitating a flow of irrigation liquid from the reservoir, through the tubing, to the catheter, the pump being connected to a power source;
   a capacitive sensor configured to measure one or more capacitive characteristics of a fluid in the tubing; and
   a logic gate arranged in series between the power source and the pump, and connected to the capacitive sensor;
wherein the capacitive sensor is configured to generate a binary output, and wherein the logic gate is configured to terminate power to the pump in accordance with receiving a binary input from the capacitive sensor being indicative of the one or more capacitive characteristics of a fluid in the tubing being indicate of gas.
2. The irrigation system according to item 1, wherein the pump is an electrical air pump configured to pressurize the reservoir to facilitate the flow of irrigation liquid.
3. The irrigation system according to any of items 1-2, wherein the capacitive sensor comprises a capacitive proximity sensor.
4. The irrigation system according to item 3, wherein the capacitive proximity sensor comprises a sensor plate arranged adjacent to the tubing.
5. The irrigation system according to item 4, wherein the capacitive proximity sensor further comprises an electrically grounded ground plate arranged adjacent to the tubing and opposite to the sensor plate to form a capacitor wherein the tubing is arranged between the sensor plate and the ground plate.
6. The irrigation system according to item 5, wherein the sensor plate and/or the ground plate are fitted according to an outer surface geometry of the tubing.
7. The irrigation system according to any of items 1-6, wherein the capacitive sensor comprises an auxiliary processor.
8. The irrigation system according to item 7, wherein the auxiliary processor is configured to generate a binary output based on the one or more characteristics of the fluid in the tubing.
9. The irrigation system according to item 8, wherein the binary output comprises a first binary output indicative of liquid in the tubing and a second binary output indicative of gas in the tubing.
10. The irrigation system according to item 9, wherein the logic gate is configured to receive the first binary output as a first binary input and to receive the second binary output as a second binary output, and wherein the logic gate is configured to switch in accordance with the received first and second binary output.
11. The irrigation system according to item 10, wherein the logic gate is configured to provide power from the power source to the pump in accordance with receiving the first binary input, and wherein the logic gate is configured to terminate power from the power source to the pump in accordance with receiving the second binary input.
12. The irrigation system according to any of items 7-11, wherein the auxiliary processor comprises a hysteresis function.
13. The irrigation system according to any of items 1-12, wherein the one or more capacitive characteristics are selected from one or more of (i) a capacitance as induced by a fluid, (ii) a permittivity of a fluid, (iii) a change of the capacitance as induced by a fluid, and (iv) a change of the permittivity.
14. A method of controlling an irrigation procedure in an irrigation system according to any of items 1-13, the method being performed in the irrigation system, such as by the capacitive sensor and the logic gate, the reservoir of the irrigation system comprising an irrigation liquid, the method comprising the steps of:
   measuring one or more capacitive characteristics of a fluid in the tubing; and
   controlling the irrigation procedure in accordance with the one or more capacitive characteristics of the fluid in the tubing.
15. The method according to item 14, wherein controlling the irrigation procedure comprises, in accordance with the one or more capacitive characteristics of the fluid in the tubing being indicative of air, generating a binary output for the logic gate, the binary output causing the logic gate to terminate power from the power source to the pump.

1. An irrigation system configured to run an irrigation procedure for irrigation of the bowels of a user, the system comprising:
a housing comprising a reservoir for containing an irrigation liquid;
a tubing providing fluid communication between the reservoir and a catheter for use with the irrigation system;
a pump for facilitating a flow of irrigation liquid from the reservoir, through the tubing, to the catheter;
a control unit comprising a processor in communication with the pump; and
a capacitive sensor in communication with the processor and configured to measure one or more capacitive characteristics of a fluid in the tubing,
wherein the processor is configured to control the irrigation procedure in accordance with the one or more capacitive characteristics of the fluid in the tubing.

2. The irrigation system according to claim 1, wherein the pump is an electrical air pump configured to pressurize the reservoir to facilitate the flow of irrigation liquid.

3. The irrigation system according to any of claims 1-2, wherein the capacitive sensor comprises a capacitive proximity sensor.

4. The irrigation system according to claim 3, wherein the capacitive proximity sensor comprises a sensor plate arranged adjacent to the tubing.

5. The irrigation system according to claim 4, wherein the capacitive proximity sensor further comprises an electrically grounded ground plate arranged adjacent to the tubing and opposite to the sensor plate to form a capacitor wherein the tubing is arranged between the sensor plate and the ground plate.

6. The irrigation system according to claim 5, wherein the sensor plate and/or the ground plate are fitted according to an outer surface geometry of the tubing.

7. The irrigation system according to any of claims 1-6, wherein the capacitive sensor comprises an auxiliary processor connected to the processor of the control unit.

8. The irrigation system according to claim 7, wherein the auxiliary processor is configured to generate a binary output based on the one or more capacitive characteristics of the fluid in the tubing.

9. The irrigation system according to claim 8, wherein the binary output comprises a first binary output indicative of liquid in the tubing and a second binary output indicative of gas in the tubing.

10. The irrigation system according to any of claims 7-9, wherein the auxiliary processor comprises a hysteresis function.

11. The irrigation system according to any of claims 1-10, wherein the one or more capacitive characteristics are selected from one or more of (i) a capacitance as induced by a fluid, (ii) a permittivity of a fluid, (iii) a change of the capacitance as induced by a fluid, and (iv) a change of the permittivity.

12. The irrigation system according to any of claims 1-11, wherein the processor is configured to terminate the irrigation procedure in accordance with the one or more capacitive characteristics of the fluid in the tubing being indicative of gas.

13. The irrigation system according to any of claims 1-12, wherein to control the irrigation procedure comprises to control the pump.

14. A method of controlling an irrigation procedure in an irrigation system according to any of claims 1-13, the method being performed by a processor of the irrigation system, the reservoir of the irrigation system comprising an irrigation liquid, the method comprising the steps of:
initiating the irrigation procedure;
measuring one or more capacitive characteristics of a fluid in the tubing; and
controlling the irrigation procedure in accordance with the one or more capacitive characteristics of the fluid in the tubing.

15. The method according to claim 14, wherein controlling the irrigation procedure comprises terminating the irrigation procedure in accordance with the one or more capacitive characteristics of the fluid in the tubing being indicative of gas.
